(19) United States
(12) Patent Application Publication
(10) Pub. No.: US 2010/0191275 A1
(43) Pub. Date: Jul. 29, 2010

(54) FILTER DEPLOYMENT DEVICE

(75) Inventors: Ari Ryan, Mountain View, CA (US); Amr Salahieh, Saratoga, CA (US)

Correspondence Address: CROMPTON, SEAGER & TUFTE, LLC, 1221 NICOLLET AVENUE, SUITE 800, MINNEAPOLIS, MN, 55403-2420, US

(73) Assignee: BOSTON SCIENTIFIC SCIMED, INC., Maple Grove, MN (US)

(21) Appl. No.: 12/360,597

(22) Filed: Jan. 27, 2009

(51) Int. Cl. A61B 17/22

(52) U.S. Cl. 606/200

(57) ABSTRACT

The invention provides a filter device associated with a guide wire, a device for deploying the filter from a containment element associated with the filter, and a method of deploying the filter from the containment element.

### TECHNICAL FIELD

This disclosure relates generally to a filter device and a device for deploying the filter. The actuation mechanism of the deployment device is well suited to the deployment of certain filter elements as well as other medical devices.

### BACKGROUND

Human blood vessels often become occluded or blocked by plaque, thrombi, other deposits, or material that reduce the blood carrying capacity of the vessel. Should the blockage occur at a critical place in the circulatory system, serious and permanent injury, and even death, can occur. To prevent this, some form of medical intervention is usually performed when significant occlusion is detected.

Several procedures are now used to open these stenosed or occluded blood vessels in a patient caused by the deposit of plaque or other material on the walls of the blood vessels. Angioplasty, for example, is a widely known procedure wherein an inflatable balloon is introduced into the occluded region. The balloon is inflated, dilating the occlusion, and thereby increasing the intraluminal diameter.

Another procedure is atherectomy. During atherectomy, a catheter is inserted into a narrowed artery to remove the matter occluding or narrowing the artery, i.e., fatty material. The catheter includes a rotating blade or cutter disposed in the tip thereof. Also located at the tip are an aperture and a balloon disposed on the opposite side of the catheter tip from the aperture. As the tip is placed in close proximity to the fatty material, the balloon is inflated to force the aperture into contact with the fatty material. When the blade is rotated, portions of the fatty material are shaved off and retained within the interior lumen of the catheter. This process is repeated until a sufficient amount of fatty material is removed and substantially normal blood flow is resumed.

In another procedure, stenosis within arteries and other blood vessels is treated by permanently or temporarily introducing a stent into the stenosed region to open the lumen of the vessel. The stent typically includes a substantially cylindrical tube or mesh sleeve made from such materials as stainless steel or nitinol. The design of the material permits the diameter of the stent to be radially expanded, while still providing sufficient rigidity such that the stent maintains its shape once it has been enlarged to a desired size.

Such percutaneous interventional procedures, i.e., angioplasty, atherectomy, and stenting, may dislodge material from the vessel walls. Some existing devices and technology use a filter for capturing the dislodged material from the bloodstream.

### SUMMARY

The present disclosure pertains to a filter deployment apparatus configured to be used in connection with an intravascular device, the filter deployment apparatus comprising a guide wire, a filter element which can be associated with the guide wire, a containment element, and a sheath disposed about the guide wire. The sheath can be slideably movable relative to the guide wire between a first position and a second position and further the sheath can include a distally extending projection capable of engaging with the filter element and/or the containment element when the sheath is in the first position, and disengaging from at least one of the filter element and the containment element when the sheath is in the second position. The apparatus can include a support element for the filter element.

The disclosure also pertains to a method of deploying a filter element, or other medical device, comprising providing a guide wire, providing a filter element which can be associated with the guide wire, providing a containment element for the filter element, providing a sheath disposed about the guide wire. The sheath can be slideably movable relative to the guide wire between a first position and a second position and further, the sheath can include a distally extending projection capable of engaging with at least one of the filter element and the containment element when the sheath is in the first position, and disengaging from at least one of the filter element and the containment element when the sheath is in the second position. Moving the sheath relative to the guide wire from the first position to the second position can disengage the distally extending projection from the filter element, and the containment element and the sheath are in the second position, deploying the filter element.

### BRIEF DESCRIPTION OF THE DRAWING

FIGS. 1A-C illustrate a filter deployment apparatus.

FIGS. 2A-E illustrate various sheaths suitable for use in the deployment apparatus

### DETAILED DESCRIPTION

The following description should be read with reference to the drawing wherein like reference numerals indicate like elements throughout the several views. The drawings, which are not necessarily to scale, are not intended to limit the scope of the claimed invention. The detailed description and drawings illustrate example embodiments of the claimed invention.

All numbers are herein assumed to be modified by the term “about.” The recitation of numerical ranges by endpoints includes all numbers subsumed within that range (e.g., 1 to 5 includes 1, 1.5, 2, 2.75, 3, 3.80, 4, and 5).

As used in this specification and the appended claims, the singular forms “a”, “an”, and “the” include the plural referents unless the content clearly dictates otherwise. As used in this specification and the appended claims, the term “or” is generally employed in its sense including “and/or” unless the content clearly dictates otherwise.

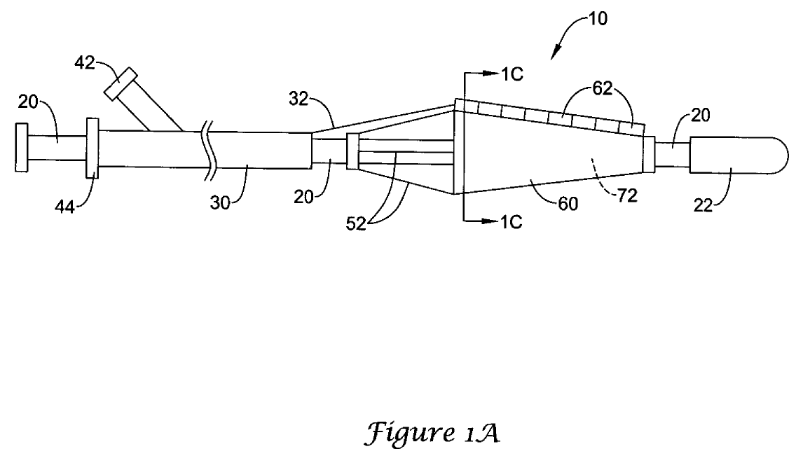

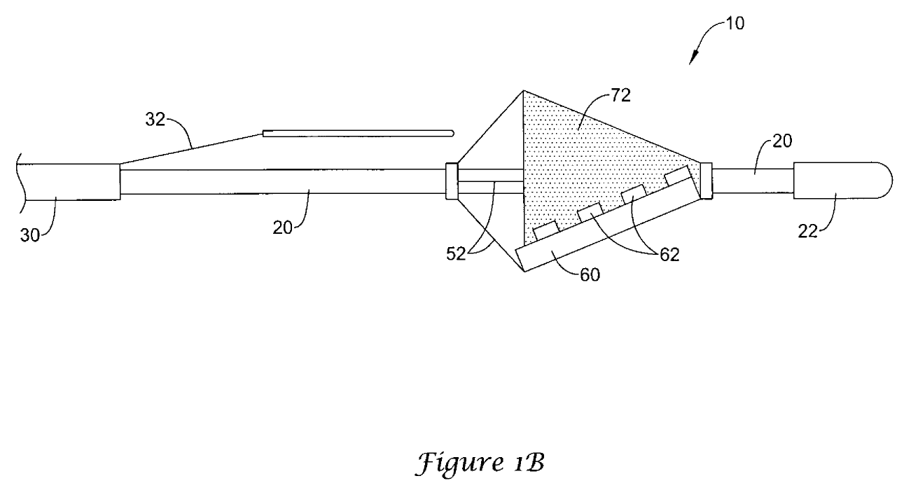

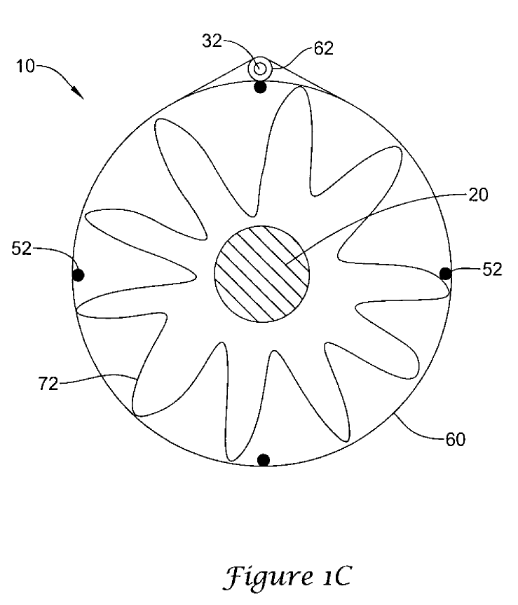

FIG. 1A illustrates a filter deployment apparatus 10 of the invention. Filter element 72 and support structure elements 52 are disposed near the distal end of guide wire 20 and enclosed in containment element 60. Containment element 60 is held closed by distally extended projection 32 of sheath 30, said distally extended projection passing through apertures 62 formed as loops alternating and mutually engaged along the edge of containment element 60. Guide wire 20 can be passed through a stenosed blood vessel guided by distal tip 22 in the conventional manner. When the enclosed filter is positioned down stream the obstruction to be removed, sheath 30 can be withdrawn proximally by pulling on proximal enlargement 44 of sheath 30 while holding guide wire 20 stationary (or visa versa). The disengages distally extended projection 32 of sheath 30 from apertures 62 in containment element 60 allowing the support structure 52 to open as shown in FIG. 1B. Outwardly biased support structure elements 52 expand and deploy filter 72 to substantially fill the lumen of the blood vessel.

FIG. 1C illustrates a transverse cross-section of FIG. 1A generally along line 1C-1C. In 1C, filter element 72 is furled within containment element 60 between support structures 52 and about guide wire 20 prior to deployment. Distally extended projection 32 passes through apertures 62 formed as loops alternating and mutually engaged along the edge of containment element 60.

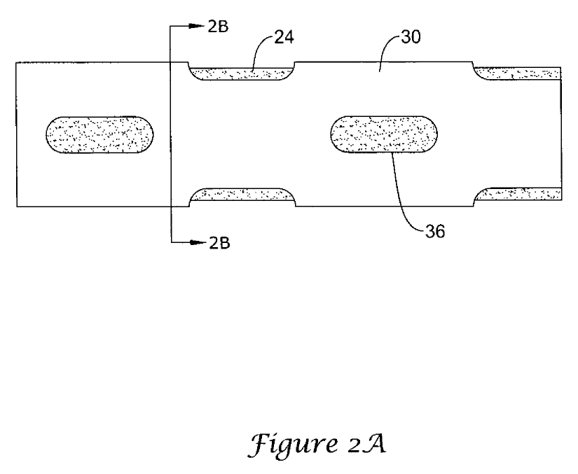

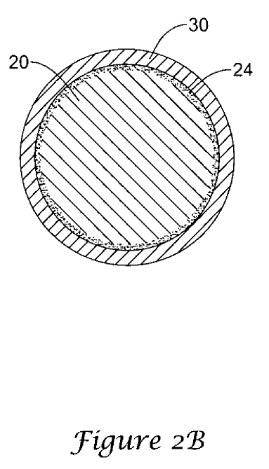

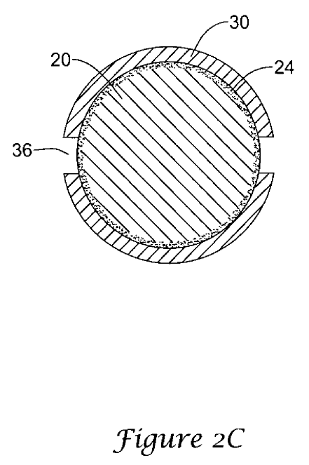

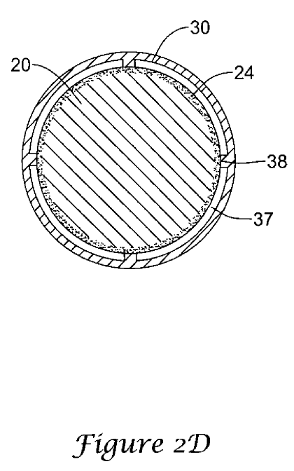

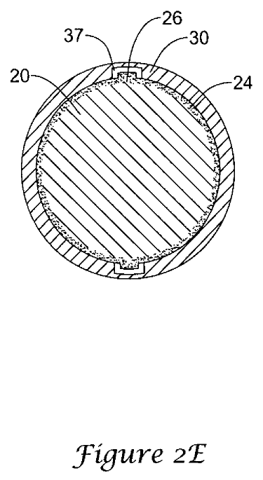

Sliding friction between the close fitting sheath 30 and guide wire 20 can be reduced in a number of ways. For example, the close fitting sheath described herein, can have a relatively large apparent contact area and may have a correspondingly large frictional force resisting motion of the sheath relative to the guide wire. The frictional drag may be mitigated by roughening the interior surface of the sheath 30 or the surface 24 of the guide wire 20 as shown in FIG. 2A. Alternately, portions of the sheath may be removed in patterns of holes and or slits 36 to further reduce contact between the sheath 30 and the guide wire surface 24 as shown in FIG. 2B. Guide wire surface roughening 24 may be combined with partial removal of the sheath 30 as illustrated in FIG. 2C, a transverse cross-section of the guide wire and sheath of FIG. 2B generally along line 2C-2C. Also, profiled extruded sheath having limited contact with guide wire 20 may be employed as illustrated in transverse section FIG. 2D. Friction between sheath 30 and guide wire 20 may be reduced, especially during relative translation, by injecting a fluid through port 42 into the lumen of the sheath and along guide wire 20 to provide a lubricated, fluid bearing.

When it is desired to limit rotation of the sheath relative to the guide wire, a sheath formed by profiled extrusion may be combined with projections formed on the guide wire as shown in FIG. 2E. Alternately, projections may be formed on the guide wire by slightly flattening the wire.

Filter element 72 can include a porous film or mesh generally in the form of a basket or cage. The porous film or mesh may have a plurality of holes, formed for example by laser drilling, sized to allow blood cells to pass freely through the filter while retaining embolic or foreign material. Filter element 72 may include one or more struts which serve to support a porous film or mesh. Such struts generally provide a shape for filter element 72 and may be biased to expand filter element 72 against the wall of the vessel in which the filter is deployed. The support structure may also provide a seal between an open end of the filter and the wall of the vessel in which filter element 72 is deployed. Filter element 72 may comprise a plurality of struts without an associated porous film or mesh.

As described above, containment element 60 can include a plurality of apertures which engage a distally extended projection of the sheath to prevent expansion of the filter element. When filter element 72 is at the desired deployment site, the sheath along with its distally extended projection is moved from a first position to a second position causing the distally extended projection to disengage from at least one aperture of containment element 60 which allows the filter element to deploy. Additionally, representative containment elements suitable for the practice of this invention are known and may be found, for example, in FIGS. 26-41 of U.S. Pat. 6,997,939, incorporated herein by reference. It should be noted that the containment elements of the reference contain the distal end of a catheter, however the structures of the containment elements themselves generally may be adapted to the current purpose. Containment element 60 can be fabricated from a variety of different materials or combination of materials, so long as the material is sufficiently strong to restrain the filter element and/or filter support structures. For examples containment element 60 may be from various types of polymeric films, such as low-density polyethylene, polyethylene terephthalate, polytetrafluoroethylene, fluorinated ethylene propylene, polyurethane, or silicone. A containment element may encompass the filter element, the support element, or both. Separate containment elements may be employed to encompass the filter element and the support elements. Portions of a containment element can overlap to bridge a gap in the containment element and are held together in the manner of a common door hinge or piano hinge with a hinge pin.

In some embodiments the distally extended project 32 of the sheath 30 may be stiffened, while in other embodiments, it may be relatively flexible. It may extend substantially along the surface of guide wire 20 or it may include one or more bends to follow the outer contour of the filter, struts, and containment element. Distally extended projection 32 associated with the sheath may be made from the same material as the sheath, or it may include other materials attached to the sheath by any of the commonly employed bonding means.

Sheath 30 may be fabricated from any convenient material having appropriate mechanical and frictional properties. Materials having a low coefficient of friction especially for those embodiments in which the sheathed guide wire will be used as a guide wire for the deployment of other medical devices such as a stent. Polyurethanes, polyethylene terephthalate, polytetrafluoroethylene, and the like may be used to form the sheath. The apparatus may be configured to provide a fluid flow within a lumen of the sheath and along the surface of the guide wire. Given the small size of the guide wire and the minimal clearance desired between the guide wire and the sheath, in some embodiments a little as a few drops of fluid may be sufficient to lubricate the interface between the guide wire and the sheath. The sheath may be sufficiently elastic to enable even a very modest fluid flow to inflate the sheath and thereby reduce contact between the guide wire and the lumen of the sheath. The fluid flow may be supplied by any convenient means such as by a syringe port on the portion of the sheath near the proximal end of the guide wire. If fluid flow is utilized it may be used while the sheath is moved from one position to another.

It should be understood that this disclosure is, in many respects, only illustrative. Changes may be made in details, particularly in matters of shape, size, and arrangement of steps without exceeding the scope of the invention. The scope of the invention is, of course, defined in the language in which the appended claims are expressed.

What is claimed is:

1. An embolic filter deployment apparatus configured to be used in connection with an intravascular device, the embolic filter deployment apparatus comprising:
a guide wire;
a filter element associated with the guide wire;
a containment element associated with the filter element; and
a sheath disposed about the guide wire, wherein the sheath is slideably movable relative to the guide wire between a first position and a second position and further wherein the sheath includes a distally extending projection capable of engaging with at least one of the filter element and the containment element when the sheath is in the first position and disengaging from at least one of the filter element and the containment element when the sheath is in the second position.

2. The filter deployment apparatus of claim 1, wherein the filter element includes a porous film or mesh.

3. The filter deployment apparatus of claim 1, wherein the filter element includes a support structure.

4. The filter deployment apparatus of claim 1, wherein the containment element at least partially surrounds the filter element in the first position.

5. The filter deployment apparatus of claim 3, wherein the containment element at least partially surrounds the support structure for the filter element in the first position.

6. The filter deployment apparatus of claim 1, wherein the containment element includes one or more apertures which engage the distally extending projection of the sheath when the sheath is in the first position and one or more apertures which are disengaged from the distally extending projection when the sheath is in the second position.

7. The filter deployment apparatus of claim 4, wherein the apertures are selected from holes, loops, hooks, grooves, and combinations thereof.

8. The filter deployment apparatus of claim 1, wherein the sheath includes more than one distally extending projection.

9. The filter deployment apparatus of claim 1, wherein the sheath has a lumen having a cross-sectional shape which differs from the cross-sectional shape of the guide wire.

10. The filter deployment apparatus of claim 1, wherein the sheath is at least partially relieved such that the contact area between the sheath and the guide wire is, on average, less than the surface area of the portion of the guide wire within the sheath.

11. The filter deployment apparatus of claim 1, wherein the apparatus is configured to provide a fluid flow within a lumen of the sheath and along the surface of the guide wire.

12. The filter deployment apparatus of claim 1, wherein when the sheath is slideably moved from a first position to a second position, it is substantially free of rotation around the guide wire.

13. The filter deployment apparatus of claim 1, wherein when the sheath is slideably moved from a first position to a second position, it also rotates around the guide wire.

14. A method of deploying an embolic filter comprising:
providing a guide wire;
providing a filter element associated with the guide wire;
providing a containment element associated with the filter element;
providing a a sheath disposed about the guide wire, wherein the sheath is slideably movable relative to the guide wire between a first position and a second position and further wherein the sheath includes a distally extending projection capable of engaging with at least one of the filter element and the containment element when the sheath is in the first position and disengaging from at least one of the filter element and the containment element when the sheath is in the second position;
moving the sheath relative to the guide wire from the first position to the second position;
disengaging the distally extending projection from at least one of the filter element and the containment element when the sheath is in the second position; and
deploying the filter element.

15. The method of claim 14, wherein the filter element includes a porous film or mesh.

16. The method of claim 14, wherein the filter element includes a support structure.

17. The method of claim 14, wherein the containment element at least partially surrounds the filter clement in the first position.

18. The method of claim 16, wherein the containment element at least partially surrounds the support structure for the filter element in the first position.

19. The method of claim 14, wherein the containment element includes one or more apertures which engage the distally extending projection of the sheath when the sheath is in the first position and one or more apertures which are disengaged from the distally extending projection when the sheath is in the second position.

20. The method of claim 19, wherein the apertures are selected from holes, loops, hooks, grooves, and combinations thereof.

21. The method of claim 14, wherein the sheath has a lumen having a cross-sectional shape which differs from the cross-sectional shape of the guide wire.

22. The method of claim 14, wherein the sheath is at least partially relieved such that the contact area between the sheath and the guide wire is, on average, less than the surface area of the portion of the guide wire within the sheath.

23. The method of claim 14, further including providing a fluid flow within a lumen of the sheath and along the surface of the guide wire during moving the sheath relative to the guide wire from the first position to the second position.

24. The method of claim 14, wherein moving the sheath from a first position to a second position also rotates around the guide wire.

25. The method of claim 14, wherein a medical device is slideably disposed over the sheath either before or after the deployment of the filter device.

26. The method of claim 14, wherein the filter element is replaced by another medical device to be deployed from the containment element.
